(19) United States
(12) Patent Application Publication
(10) Pub. No.: US 2010/0152269 A1
(43) Pub. Date: Jun. 17, 2010

(54) MODIFIED-RELEASE FORMULATIONS OF AZABICYCLO DERIVATIVES

(75) Inventors: Anant Ramesh Ketkar, Haryana (IN); Pratik Kumar, Bihar (IN); Ashok Rampal, Haryana (IN)

Correspondence Address: Ranbaxy Inc., Intellectual Property Department, 600 College Road East, PRINCETON, NJ, 08540, US

(73) Assignee: RANBAXY LABORATORIES LIMITED, Gurgaon, Haryana (IN)

(21) Appl. No.: 12/519,479

(22) PCT Filed: Dec. 21, 2007
(86) PCT No.: PCT/IB07/55299

§ 371 Date: Dec. 21, 2009

(30) Foreign Application Priority Data
Dec. 26, 2006 (IN) 2751/DEL/2006

(51) Int. Cl. A61K 31/403; A61P 11/00; A61P 1/00; A61P 13/00

(52) U.S. Cl. 514/412

(57) ABSTRACT

The present invention discloses modified-release oral dosage forms of an azabicyclo derivative or its pharmaceutically acceptable salts, solvates, esters, enantiomers, diastereomers, N-oxides and polymorphs; and processes for the preparation thereof. The modified release formulation comprises an azabicyclo derivative, at least one rate-controlling polymer and at least one pharmaceutically acceptable excipient which provides therapeutically effective plasma levels of the active ingredient for a period of up to 24 hours.

### TECHNICAL FIELD OF THE INVENTION

The present invention relates to modified-release oral dosage forms of an azabicyclo derivative or its pharmaceutically acceptable salts, solvates, esters, enantiomers, diastereomers, N-oxides and polymorphs; and processes for the preparation thereof.

### BACKGROUND OF THE INVENTION

PCT Application WO 2004/005252 discloses azabicyclo derivatives as muscarinic receptor antagonists, and particularly discloses Compound (I), (2R)-(1α,5α,6α)-N-[3-azabicyclo[3.1.0]hexyl-6-(aminomethyl)-yl]-2-hydroxyl-2 cyclopentyl-2-phenyl acetamide. The application further relates to processes for the preparation of this and other compounds.

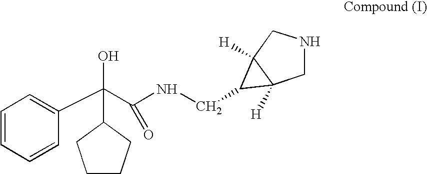

Further, PCT Application WO 2006/003587 describes a solid dosage form of Compound (I) having excellent content uniformity which can be prepared by wet granulating a blend of excipients with a solution of Compound (I).

Compound (I) is a muscarinic receptor antagonist with high affinity towards M3 receptors and may be useful as a safe and effective therapeutic or prophylactic agent for the treatment or prophylaxis of an animal or a human suffering from a disease or disorder of the respiratory, urinary and gastrointestinal systems, wherein the disease or disorder is mediated through, or associated with, muscarinic receptors. The diseases or disorders may include diseases and disorders of the (a) respiratory system such as bronchial asthma, chronic obstructive pulmonary disorders (COPD), pulmonary fibrosis, and the like; (b) urinary system which include urinary incontinence, lower urinary tract symptoms (LUTS), etc.; and (c) gastrointestinal system such as irritable bowel syndrome, obesity, diabetes and gastrointestinal hyperkinesis, and the like.

Compound (I) is a highly potent compound and can be administered at a very low dosage level. Compound (I) undergoes metabolism primarily by glucuronidation along with oxidation. The metabolites have comparable activity as that of the parent compound and are considered to contribute significantly to the therapeutic efficacy of Compound (I) itself. On the other hand, the non-specific selectivity of these metabolites to muscarinic receptor binding sites is thought to be a major cause for side effects associated with Compound (I).

Conventional immediate release dosage forms of Compound (I) are preferably administered twice a day. However, such dosage forms are associated with usual antimuscarinic side effects, namely dry mouth, blurred vision, confusion and constipation, especially at higher doses. This may lead to discontinuation of treatment with Compound (I). Such adverse effects can be attributed to the high peak plasma levels of Compound (I) and its metabolites.

Consequently, there arises a need for the preparation of a dosage form of Compound (I) which would provide a controlled-release profile for an extended period of time that would reduce the peak plasma concentration of the drug and in turn its metabolites. This would lead to alleviation of the unwanted side-effects associated with a conventional immediate-release dosage form without compromising the therapeutic efficacy. Such a preparation would also decrease the frequency of administration to once-daily, thereby improving patient compliance.

Thus, according to the present invention, there is provided a modified-release formulation, for the treatment and prophylaxis of diseases and disorders responsive to muscarinic receptor activity, comprising a therapeutically effective amount of Compound (I), or a pharmaceutically acceptable salt, solvate, ester, enantiomer, diastereomer, N-oxide or polymorph thereof.

### BRIEF DESCRIPTION OF THE DRAWING

FIG. 1 is a graph of the mean concentration time profile of Compound (I) following oral administration of modified release tablets 0.1 mg OD (Tablets of Examples 1 and 2, designated as A and B respectively) and Immediate Release capsules 0.05 mg (0.025×2) capsules (designated as R) BID in healthy human subjects.

### SUMMARY OF THE INVENTION

In one general aspect modified-release formulations are provided, comprising a therapeutically effective amount of Compound (I):

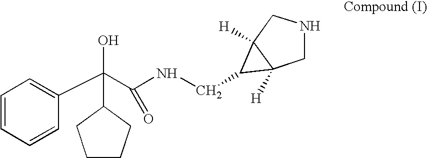

at least one rate-controlling polymer, and one or more pharmaceutically acceptable excipients.

In another general aspect, modified-release formulations are provided, comprising Compound (I); at least one rate-controlling polymer; and one or more pharmaceutically acceptable excipients, such that, the said formulation exhibits at least one of the following in-vitro dissolution profiles when measured in a USP type II apparatus, at 50 rpm, at a temperature of 37° C.±0.5° C. in 900 mL of 0.1N hydrochloric acid:

(i) at least about 25% drug released in 2 hours;

(ii) at least about 40% drug released in 4 hours; or

(iii) at least about 65% drug released in 8 hours.

In another general aspect, modified-release formulations are provided, comprising Compound (I); at least one hydrophilic polymer; and one or more pharmaceutically acceptable excipients selected from diluents, binders, glidants and lubricants.

In another general aspect, modified-release formulations are provided, comprising Compound (I); at least one water-swellable cellulosic polymer; and one or more pharmaceutically acceptable excipients selected from diluents, binders, glidants and lubricants.

In another general aspect, modified-release formulations are provided, comprising Compound (I); hydroxypropyl methylcellulose; and one or more pharmaceutically acceptable excipients selected from diluents, binders, glidants and lubricants.

In another general aspect, processes for the preparation of modified-release formulations of Compound (I) are provided, wherein the processes comprise:
a) blending one or more pharmaceutically acceptable excipients selected from diluents and binders;
b) granulating the blend with a solution of Compound (I);
c) drying and sizing the granules of step (b);
d) blending the dried granules of step (c) with one or more rate-controlling polymer;
e) mixing the blend of step (d) with one or more pharmaceutically acceptable excipients selected from diluents, lubricants and glidants; and
f) processing the blend of step (e) into a dosage form.

In another general aspect, processes for the preparation of modified-release formulations of Compound (I) are provided, wherein the processes comprise:
a) blending one or more pharmaceutically acceptable excipients selected from diluents and binders;
b) granulating the blend with a solution of Compound (I);
c) drying and sizing the granules of step (b);
d) blending the dried granules of step (c) with one or more hydrophilic polymer;
e) mixing the blend of step (d) with one or more pharmaceutically acceptable excipients selected from diluents, lubricants and glidants; and
f) processing the blend of step (e) into a dosage form.

In another general aspect, processes for the preparation of modified-release formulations of Compound (I) are provided, wherein the processes comprise:
a) blending one or more pharmaceutically acceptable excipients selected from diluents and binders;
b) granulating the blend with a solution of Compound (I);
c) drying and sizing the granules of step (b);
d) blending the dried granules of step (c) with one or more water-swellable cellulosic polymer;
e) mixing the blend of step (d) with one or more pharmaceutically acceptable excipients selected from diluents, lubricants and glidants; and
f) processing the blend of step (e) into a dosage form.

In another general aspect, processes for the preparation of modified-release formulations of Compound (I) are provided, wherein the processes comprise:
a) blending one or more pharmaceutically acceptable excipients selected from diluents and binders;
b) granulating the blend with a solution of Compound (I);
c) drying and sizing the granules of step (b);
d) blending the dried granules of step (c) with hydroxypropyl methylcellulose;
e) mixing the blend of step (d) with one or more pharmaceutically acceptable excipients selected from diluents, lubricants and glidants; and
f) processing the blend of step (e) into a dosage form.

In another general aspect, modified-release formulations, are provided, comprising Compound (I); at least one rate-controlling polymer and one or more pharmaceutically acceptable excipients, wherein the formulation provides therapeutically effective plasma levels of Compound (I) for a period of up to about 24 hours.

In another general aspect, modified-release formulations are provided, comprising Compound (I); at least one hydrophilic polymer and one or more pharmaceutically acceptable excipients, wherein the formulation provides therapeutically effective plasma levels of Compound (I) for a period of up to about 24 hours.

In another general aspect, modified-release formulations are provided, comprising Compound (I); at least one water-swellable cellulosic polymer and one or more pharmaceutically acceptable excipients, wherein the formulation provides therapeutically effective plasma levels of Compound (I) for a period of up to about 24 hours.

In another general aspect, modified-release formulations are provided comprising Compound (I); hydroxypropyl methylcellulose and one or more pharmaceutically acceptable excipients, wherein the formulation provides therapeutically effective plasma levels of Compound (I) for a period of up to about 24 hours.

### DETAILED DESCRIPTION OF THE INVENTION

The term “modified-release formulation” as referred to herein means an oral pharmaceutical dosage form which releases therapeutically active medicament at a controlled rate such that therapeutically beneficial blood levels of the medicament are maintained over an extended period of time, and includes prolonged-, controlled-, extended-, delayed- and sustained-release formulations. Such formulations include matrix systems, multiparticulate systems, ion-exchange resin beads, pulsatile devices, multilayered tablets, osmotic systems, reservoir-based systems and membrane-controlled systems. Particularly preferred are matrix-based systems. The formulation may be in the form of granules, tablets, pellets or capsules. In one embodiment the formulation can be a tablet.

Compound (I) as described in the present invention includes pharmaceutically acceptable salts, solvates, esters, enantiomers, diastereomers, N-oxides and polymorphs of Compound (I). Compound (I) as described herein is used in a therapeutically effective amount in the solid dosage forms. The term “therapeutically effective amount” describes a dose of a compound (I) that is effective for the treatment or prophylaxis of a disease or disorder of the respiratory, urinary and/or gastrointestinal systems related to muscarinic receptor activity. The dose may range from about 0.01 μg to about 20 mg of compound (I) by weight of the formulation.

The rate-controlling polymer may be either a hydrophilic or hydrophobic polymer; and particularly polymers that swell in aqueous media. The amount of polymer relative to Compound (I) depends upon the rate of drug release required and also upon the type and molecular weight of the polymer and other excipients present in the formulation. Hydrophilic polymers suitable for use in the modified release formulation include one or more water-swellable cellulosic polymers such as hydroxypropyl methylcellulose, methylcellulose, hydroxymethyl cellulose carboxymethyl cellulose, sodium carboxymethyl cellulose, hydroxypropylcellulose and hydroxyethylcellulose. Other hydrophilic polymers include natural or partially or totally synthetic hydrophilic gums such as acacia, gum tragacanth, locust bean gum, guar gum, karaya gum; proteinaceous substances such as agar, pectin, carrageen, and alginates; polyvinylpyrrolidone; vinyl acetate copolymers; starch and starch based polymers; polysaccharides; methacrylic acid copolymers; maleic anhydride/methyl vinyl ether copolymers; carboxypolymethylene; gelatin; casein, zein; bentonite; magnesium aluminium silicate; polyethylene oxide and other hydrophilic polymers known to those skilled in the art or a derivative or a mixture thereof.

In a particular embodiment, the preferred hydrophilic polymers are the hydroxypropyl methylcelluloses. The hydroxypropyl methylcellulose can be of different viscosity grades having the viscosity from about 100 cps to about 100,000 cps. Suitable types are sold, for example, under the trade name of Methocel by The Dow Chemical Company such as Methocel K4MCR, Methocel K15MCR, and Methocel K100MCR.

Besides the above, hydrophobic polymers which may be used include, for example, polymers such as ethyl cellulose, cellulose acetate, acrylic acid polymers and copolymers, high molecular weight polyvinyl alcohols and waxes such as fatty acids and glycerides.

The amount of rate-controlling polymer in the dosage form may vary from about 5% to about 80% by weight of the composition, in particular from about 5% to about 70%, more particularly from about 5% to about 50% by weight of the modified-release formulation.

The modified-release formulation of Compound (I) as described herein may further comprise one or more pharmaceutically acceptable excipients, such as diluent(s), binder(s), lubricant(s) and glidant(s).

Diluents that may be used may include, but are not limited to, saccharides like lactose, dextrose, sucrose, fructose, maltose; sugars like mannitol, erythritol, sorbitol, xylitol and lactitol; cellulose derivatives like powdered cellulose, microcrystalline cellulose; dicalcium phosphate, tribasic calcium phosphate, calcium sulphate, calcium carbonate, kaolin and the like. The diluent may comprise from about 50% to about 95% by weight of the modified-release formulation. In one particular embodiment, the diluent can be microcrystalline cellulose.

Binders that may be used include, but are not limited to, starch derivatives like corn starch and pregelatinized starch; cellulose ethers such as carboxymethyl cellulose, methylcellulose, hydroxypropyl cellulose, hydroxypropyl methylcellulose; carboxy vinyl polymers like carbomers; acrylates such as Eudragits; polyvinylpyrrolidone, polyvinylpyrrolidone/vinyl acetate copolymer; xanthan gum, guar gum and other such materials routinely used in the art of solid dosage form manufacturing. The binder may be present in an amount varying from about 1% to about 15% by weight of the modified-release formulation.

Lubricants can include, for example, magnesium stearate, calcium stearate, zinc stearate, sodium stearyl fumarate, powdered stearic acid, magnesium oleate, calcium palmitate, potassium laureate, sodium suberate, vegetable oil, mineral oil and the like. Glidants may be selected from talc, colloidal silicon dioxide, corn starch and the like. The lubricant and glidants may be used in a concentration of about 0.1% to about 2% by weight of the modified-release formulation. In another embodiment, the lubricant and glidants used can be colloidal anhydrous silica, magnesium stearate and talc. The composition may also include additional excipients like sweeteners, flavouring agents and colouring agents.

The modified-release formulation comprising Compound (I) may be prepared using a wet granulation technique to ensure content uniformity. The solution of Compound (I) may be prepared in a suitable solvent such as water, or a mixture of water and a non-aqueous solvent. Pharmaceutically acceptable excipients consisting of one or more diluent and binder are blended and subsequently granulated with the solution of Compound (I). The mixture is kneaded until granulation is complete and Compound (I) forms an adsorbate with the blend of one or more of the excipients. The granules are then dried and passed through a screen. The dried granules are then blended with one or more rate-controlling polymer and mixed with one or more diluent(s), lubricant(s) or glidant(s). The final blend may further be compressed into tablets or filed into capsules.

In one embodiment, the modified-release formulation comprising Compound (I) may be prepared by-wet granulation process. The pharmaceutically-acceptable excipient, such as diluent may be granulated with a solution of Compound (I) prepared in water. The granules so formed may be dried and mixed with one or more rate-controlling polymer and further mixed with one or more of diluent, lubricant and glidant and compressed into a tablet.

In another embodiment, the modified-release formulation comprising Compound (I) may be prepared by granulating a pharmaceutically-acceptable excipient, such as diluent with a solution of Compound (I) prepared in water. The granules so formed may be dried and mixed with one or more water-swellable cellulosic polymer and further mixed with one or more of diluent, lubricant and glidant and compressed into a tablet.

In another embodiment, the modified-release formulation comprising Compound (I) may be prepared by granulating a pharmaceutically acceptable excipient, such as diluent with a solution of Compound (I) prepared in water. The granules so formed may be dried and mixed with hydroxypropyl methylcellulose and further mixed with one or more of diluent, lubricant and glidant and compressed into a tablet.

Alternately, non-aqueous granulation, spray granulation and fluidized-bed granulation techniques can also be used to prepare such formulations.

Modified-release formulation of Compound (I) and process for the preparation thereof described herein can be further illustrated by the following examples but these do not limit the scope of invention.

### Examples 1 and 2

| | | Quantity in mg/tablet | |
|---|---|---|---|
| S/N | Ingredients | Example 1 | Example 2 |
| 1. | Compound (I) hydrochloride | 0.112 | 0.112 |
| | (equivalent to 0.1 mg Compound (I) base) | | |
| 2. | Microcrystalline Cellulose | 175.888 | 125.888 |
| 3. | Hydroxypropyl methylcellulose | 70.000 | 120.000 |
| 4. | Purified Talc | 1.250 | 1.250 |
| 5. | Colloidal Anhydrous Silica | 1.000 | 1.000 |
| 6. | Magnesium Stearate | 1.750 | 1.750 |
| 7. | Purified Water | q.s. | q.s. |
| | Total Weight (in mg) | 250 | 250 |

Procedure: Compound (I) was dissolved in purified water. Microcrystalline cellulose (MCC) was sifted and blended in a rapid-mixer granulator. The above blend was granulated with the solution of Compound (I) with continuous mixing at slow impeller speed. The adsorbate granules of Compound (I)-MCC were dried in a fluidized bed dryer at 60° C. The dried adsorbate granules of Compound (I)-MCC so formed were mixed with hydroxypropyl methylcellulose in a non-shear blender. The blended adsorbate granules were finally sifted and mixed with talc, colloidal silicon dioxide and magnesium stearate. The final blend was compressed into tablets using appropriate tooling.

Tablets of Examples 1 and 2 were subjected to dissolution studies in a USP II apparatus in 0.1N HCl (900 mL) using Japanese sinkers. The temperature and agitation were set at 37° C.±0.5° C. and 50 rpm, respectively. An aliquot of sample was withdrawn at predetermined time intervals and replaced with an equal amount of fresh media. Samples were processed and analysed suitably. Dissolution profiles of these tablets are provided in Table 1.

TABLE 1
In-vitro release pattern of Compound (I) from modified release tablets
prepared as per composition of Examples 1 and 2 in USP II apparatus
in 900 mL of 0.1N HCl at 50 rpm using Japanese sinkers.

| | |
|---|---|
| Time | Percent of Compound (I) released |

| (h) | Example 1 | Example 2 |
|---|---|---|
| 1 | 26 | 19 |
| 2 | 42 | 33 |
| 4 | 64 | 51 |
| 6 | 77 | 65 |
| 8 | 86 | 75 |
| 10 | 91 | 82 |
| 12 | 95 | 87 |
| 16 | 96 | 95 |
| 20 | — | 99 |
| 24 | — | 100 |

### Bioavailability Study:

The modified-release tablets of Example 1 and 2 (test) were subjected to a bioavailability study in comparison with an immediate-release formulation of Compound (I) [0.025 mg capsule manufactured by Ranbaxy Research Laboratories, India] as a reference, in an open label, randomized, three-treatment, three-period, three-sequence, crossover bioavailability study in 15 healthy volunteers under fasting conditions. The immediate release 0.05 mg (0.025×2) capsules of Compound (I) (BID) at 12 two consecutive hourly intervals and tablets prepared as per Examples 1 and 2 were given once. The mean Tₘₐₓ, Cₘₐₓ, mean AUC₀₋₂₄ and mean AUCₗₐₛₜ of the reference and test products is illustrated in Table 2.

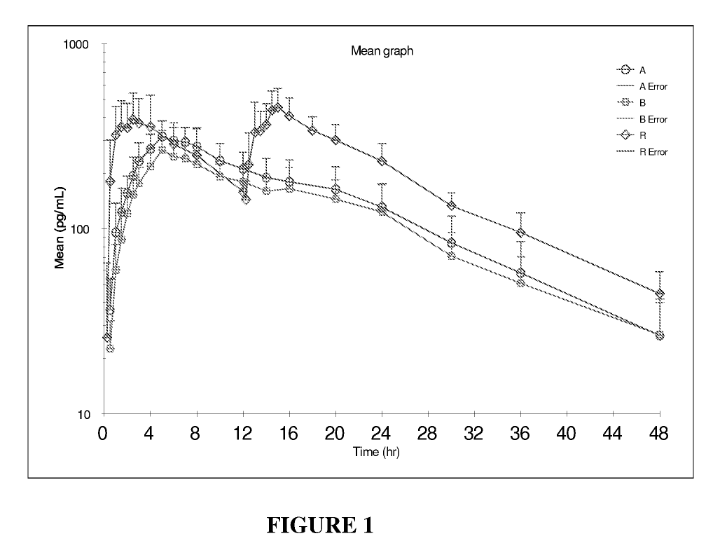

Mean drug concentration in healthy human subjects with respect to time was compared between tablets prepared as per Examples 1 (represented as A in FIG. 1) and 2 (represented as B in FIG. 1) (administered once) and immediate release 0.05 mg (0.025×2) capsules (represented as R In FIG. 1) of Compound (I) and is illustrated graphically in FIG. 1.

TABLE 2
Summary of pharmacokinetic parameters (mean ± SD) and statistics for Compound
(I) modified release tablets (Example 1 and 2) and immediate release capsules.

| | Statistics | | | |
|---|---|---|---|---|
| | Tₘₐₓ | Cₘₐₓ | AUCₗₐₛₜ | AUC₀₋₂₄ |
| Treatment | (h) | (pg/mL) | (pg · h/mL) | (pg · h/mL) |
| Immediate Release capsule | 2.5 | 434 ± 164 | 9751 ± 2287 | 10450 ± 2461 |
| Tablets of Example 1 | 5.50 | 329 ± 64 | 6368 ± 1668 | 6811 ± 1901 |
| Tablets of Example 2 | 5.0 | 281 ± 63 | 5420 ± 963 | 5932 ± 1263 |
| T/R Ratio (%): | | | | |
| Tablets of Example 1 Vs | — | 82.70 | 66.21 | 65.87 |
| Immediate Release capsule | | | | |
| Tablets of Example 2 Vs | | 66.93 | 56.63 | 57.56 |
| Immediate Release capsule | | | | |
| 90% Confidence Interval: | | | | |
| Tablets of Example 1 Vs | — | 72.04-94.04 | 57.79-75.86 | 57.44-75.54 |
| Immediate Release capsule | | | | |
| Tablets of Example 2 Vs | | 58.67-76.37 | 49.73-64.48 | 50.50-65.60 |
| Immediate Release capsule | | | | |

We claim:

1. A modified-release formulation comprising a therapeutically effective amount of a Compound (I): 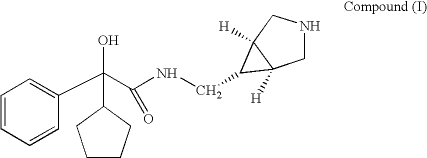
at least one rate-controlling polymer, and one or more pharmaceutically acceptable excipients.

2. The modified-release formulation according to claim 1 wherein the modified release formulation exhibits at least one of the following in-vitro dissolution profiles, when measured in a USP type II dissolution apparatus, at 50 rpm, at a temperature of 37° C.±0.5° C. in 900 mL of 0.1N hydrochloric acid:
(i) at least about 25% drug released in 2 hours;
(ii) at least about 40% drug released in 4 hours; or
(iii) at least about 65% drug released in 8 hours.

3. The modified-release formulation according to claim 1 wherein Compound (I) comprises from about 0.01 μg to about 20 mg by weight of the formulation.

4. The modified-release formulation according to claim 1 wherein the rate controlling polymer comprises from about 5% to about 80% by weight of the formulation.

5. The modified-release formulation according to claim 1 wherein the rate controlling polymer is selected from hydrophilic or hydrophobic polymer.

6. The modified-release formulation according to claim 5 wherein the hydrophilic polymer is a water swellable cellulosic polymer selected from hydroxypropyl methylcellulose, hydroxymethylcellulose, carboxymethyl cellulose, sodium carboxymethylcellulose, methylcellulose, hydroxypropylcellulose and hydroxyethylcellulose.

7. The modified-release formulation according to claim 5 wherein the hydrophilic polymer is selected from polyvinylpyrrolidone; vinyl acetate copolymers; alginate, xanthan gum, guar gum; starch and starch based polymers; polyethylene oxide; methacrylic acid copolymers and maleic anhydride/methyl vinyl ether copolymers and derivatives.

8. The modified-release formulation according to claim 1 wherein one or more pharmaceutically acceptable excipients are selected from diluents, binders, lubricants and glidants as herein described.

9. A process for the preparation of a modified release formulation of Compound (I) according to claim 1 wherein the process comprises:
a) blending one or more pharmaceutically acceptable excipients selected from diluents and binders;
b) granulating the blend with a solution of Compound (I);
c) drying and sizing the granules of step (b);
d) blending the dried granules of step (c) with one or more rate-controlling polymer;
e) mixing the blend of step (d) with one or more pharmaceutically acceptable excipients selected from diluents, lubricants and glidants; and
f) processing the blend of step (e) into a dosage form.

10. The modified-release formulation of Compound (I) and process for the preparation thereof substantially as described and illustrated by examples herein.
